(19) United States
(12) Patent Application Publication
(10) Pub. No.: US 2021/0287796 A1
(43) Pub. Date: Sep. 16, 2021

(54) SYSTEM AND METHOD FOR GENERATING RECOMMENDATIONS

(71) Applicant: HCL AMERICA, INC., Sunnyvale, CA (US)

(72) Inventor: Akshat JOSHI, Sunnyvale, CA (US)

(73) Assignee: HCL AMERICA, INC., Sunnyvale, CA (US)

(21) Appl. No.: 16/819,083

(22) Filed: Mar. 14, 2020

(51) Int. Cl. G16H 50/20; G16H 15/00; G16H 50/30

(52) U.S. Cl. CPC G16H 50/20; G16H 50/30; G16H 15/00

(57) ABSTRACT

The present disclosure relates to system(s) and method(s) for generating recommendations for a patient and a doctor. The method comprises capturing voice data comprising one or more recordings of the patient. Further, the method comprises generating a frequency spectrum for each recording from the one or more recordings. Furthermore, the method comprises analyzing the voice data and the frequency spectrum based on one of a spectral density, a coherence, a bandwidth, a signal to noise ratio, and an impedance, in order to generate analysed data. The method further comprises generating one or more recommendations for the patient and the doctor based on comparing the analysed data with historical data associated with one or more patients, and the frequency spectrum.

### CROSS-REFERENCE TO RELATED APPLICATIONS AND PRIORITY

The present application does not claim priority from any patent application.

### TECHNICAL FIELD

The present disclosure in general relates to the field of a predictive analytics in healthcare. More particularly, the present invention relates to a system and method for generating recommendations for a patient and a doctor.

### BACKGROUND

Generally, animals and children are not able to speak, and tell any health issue they are facing. In this case, if the parents think that child is not stable, they take the child to a doctor. In this case, doctor attends the patient i.e. the child, accordingly based on the observation give treatment to the patient. Sometime the doctor gives discharge to the patient once the patient is stabilised. However, the patient may face the same issue again after going home, or after getting discharge. This results in creating lot of issues between the doctor and the parents. Many times, the parents or the guardians of the patient sue the doctor or the hospital for their lack of informed decision. Nowadays, few hospitals use their own systems that tracks health data of the patients. However, such systems only store the health data, that may be used by the hospital for any legal consequences as a proof.

### SUMMARY

Before the present systems and methods for generating recommendations, is described, it is to be understood that this application is not limited to the particular systems, and methodologies described, as there can be multiple possible embodiments which are not expressly illustrated in the present disclosure. It is also to be understood that the terminology used in the description is for the purpose of describing the particular versions or embodiments only, and is not intended to limit the scope of the present application. This summary is provided to introduce concepts related to systems and method for generating the recommendations. This summary is not intended to identify essential features of the claimed subject matter nor is it intended for use in determining or limiting the scope of the claimed subject matter.

In one implementation, a method for generating recommendations for a patient and a doctor is illustrated. The method comprises capturing data associated with a health of a patient. The data comprises voice data comprising one or more recordings of the patient. The method further comprises generating a frequency spectrum for each recording from the one or more recordings. The frequency spectrum is generated using a Fourier transformation method and python libraries. The method further comprises analyzing the voice data and the frequency spectrum based on one of a spectral density, a coherence, a bandwidth, a signal to noise ratio, and an impedance. In one aspect, analysed data is generated based on the analysis. Further, the method comprises generating one or more recommendations for the patient and the doctor based on comparing the analysed data with historical data associated with one or more patients, and the frequency spectrum.

In another implementation, a system for generating recommendations for a patient and a doctor is illustrated. The system comprises a memory and a processor coupled to the memory, further the processor is configured to execute instructions stored in the memory. In one embodiment, the processor may execute instructions stored in the memory for capturing data associated with a health of a patient. The data comprises voice data comprising one or more recordings of the patients. Further, the processor may execute instructions stored in the memory for generating a frequency spectrum for each recording from the one or more recordings. The frequency spectrum is generated using a Fourier transformation method and python libraries. Furthermore, the processor may execute instructions stored in the memory for analyzing the voice data and the frequency spectrum based on one of a spectral density, a coherence, a bandwidth, a signal to noise ratio, and an impedance. In one aspect, analysed data is generated based on the analysis. The processor may further execute the instructions stored in the memory for generating one or more recommendations for the patient and the doctor based on comparing the analysed data with historical data, and the frequency spectrum.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to refer like features and components.

FIG. 1 illustrates a network implementation of a system for generating recommendations, in accordance with an embodiment of the present subject matter.

FIG. 2 illustrates the system for generating the recommendations, in accordance with an embodiment of the present subject matter.

FIGS. 3A and 3B illustrates a method for generating the recommendations, in accordance with an embodiment of the present subject matter.

### DETAILED DESCRIPTION

Some embodiments of the present disclosure, illustrating all its features, will now be discussed in detail. The words “including”, “comprising”, “consisting”, “containing”, and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a”, “an” and “the” include plural references unless the context clearly dictates otherwise. Although any systems and methods similar or equivalent to those described herein can be used in the practice or testing of embodiments of the present disclosure, the exemplary, systems and methods for generating recommendations are now described. The disclosed embodiments of the system and method for generating the recommendations are merely exemplary of the disclosure, which may be embodied in various forms.

Various modifications to the embodiment will be readily apparent to those skilled in the art and the generic principles herein may be applied to other embodiments. However, one of ordinary skill in the art will readily recognize that the present disclosure for generating recommendations is not intended to be limited to the embodiments illustrated, but is to be accorded the widest scope consistent with the principles and features described herein.

In one embodiment, a method for generating recommendations is disclosed. In the embodiment, voice data associated with a health of a patient may be captured. The voice data may comprise one or more recordings of the patient. Further, a frequency spectrum for each recording from the one or more recordings may be generated. The frequency spectrum may be generated using a Fourier transformation method and python libraries. The voice data and the frequency spectrum may be further analysed based on one of a spectral density, a coherence, a bandwidth, a signal to noise ratio, and an impedance. Based on the analysis, analysed data may be generated. Further, one or more recommendations for the patient and the doctor may be generated based on comparing the analysed data with historical data associated with one or more patients, and the frequency spectrum.

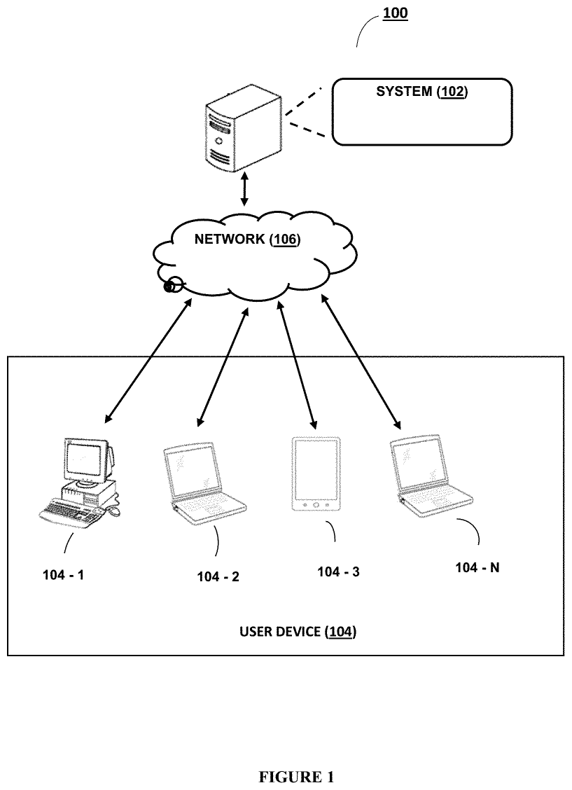

Referring now to FIG. 1, a network implementation 100 of a system 102 for generating recommendations is disclosed. Although the present subject matter is explained considering that the system 102 is implemented on a server, it may be understood that the system 102 may also be implemented in a variety of computing systems, such as a laptop computer, a desktop computer, a notebook, a workstation, a mainframe computer, a server, a network server, and the like. In one implementation, the system 102 may be implemented over a cloud network. Further, it will be understood that the system 102 may be accessed by multiple users through one or more user devices 104-1, 104-2 . . . 104-N, collectively referred to as user device 104 hereinafter, or applications residing on the user device 104. Examples of the user device 104 may include, but are not limited to, a portable computer, a personal digital assistant, a handheld device, and a workstation. The user device 104 may be communicatively coupled to the system 102 through a network 106.

In one implementation, the network 106 may be a wireless network, a wired network or a combination thereof. The network 106 may be implemented as one of the different types of networks, such as intranet, local area network (LAN), wide area network (WAN), the internet, and the like. The network 106 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further, the network 106 may include a variety of network devices, including routers, bridges, servers, computing devices, storage devices, and the like.

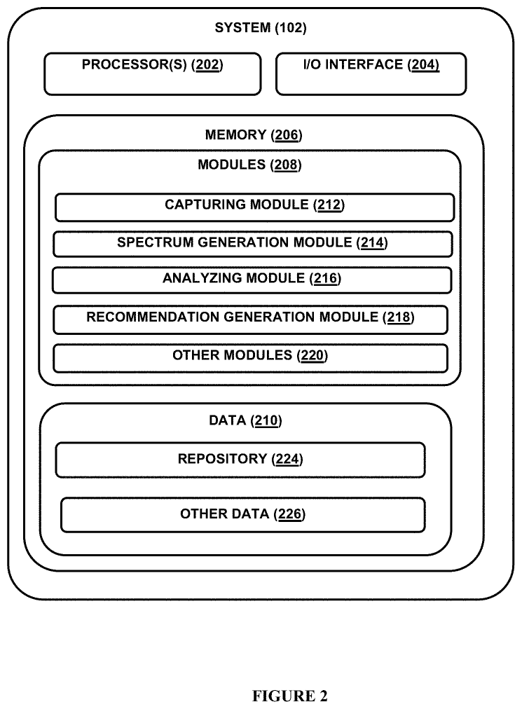

Referring now to FIG. 2, the system 102 for generating recommendations is illustrated in accordance with an embodiment of the present subject matter. In one embodiment, the system 102 may include at least one processor 202, an input/output (I/O) interface 204, and a memory 206. The at least one processor 202 may be implemented as one or more microprocessors, microcomputers, microcontrollers, digital signal processors, central processing units, state machines, logic circuitries, and/or any devices that manipulate signals based on operational instructions. Among other capabilities, at least one processor 202 may be configured to fetch and execute computer-readable instructions stored in the memory 206.

The I/O interface 204 may include a variety of software and hardware interfaces, for example, a web interface, a graphical user interface, and the like. The I/O interface 204 may allow the system 102 to interact with the user directly or through the user device 104. Further, the I/O interface 204 may enable the system 102 to communicate with other computing devices, such as web servers and external data servers (not shown). The I/O interface 204 may facilitate multiple communications within a wide variety of networks and protocol types, including wired networks, for example, LAN, cable, etc., and wireless networks, such as WLAN, cellular, or satellite. The I/O interface 204 may include one or more ports for connecting a number of devices to one another or to another server.

The memory 206 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. The memory 206 may include modules 208 and data 210.

The modules 208 may include routines, programs, objects, components, data structures, and the like, which perform particular tasks, functions or implement particular abstract data types. In one implementation, the module 208 may include capturing module 212, a spectrum generation module 214, an analysing module 216, a recommendation generation module 218, and other modules 220. The other modules 220 may include programs or coded instructions that supplement applications and functions of the system 102.

The data 210, amongst other things, serve as a repository for storing data processed, received, and generated by one or more of the modules 208. The data 210 may also include a repository 224, and other data 226. In one embodiment, the other data 226 may include data generated as a result of the execution of one or more modules in the other modules 222.

In one implementation, a user may access the system 102 via the I/O interface 204. The user may be registered using the I/O interface 204 in order to use the system 102. In one aspect, the user may access the I/O interface 204 of the system 102 for obtaining information, providing input information or configuring the system 102.

In one embodiment, the capturing module 212 may be configured to capture data associated with a health of a patient. The data may be captured in real-time. The data may correspond to voice data comprising one or more recordings of the patient. The voice data may be referred as an audio file. The one or more recordings may be recorded using a recording studio. The recording studio may use a standard instrument with standard configurations to record the one or more recordings. In one aspect, the one or more recordings may be stored at the repository 224. The one or more recordings may be saved in a specific format such as ‘.WAV’ format.

Further, the spectrum generation module 214 may generate a frequency spectrum for each recording, from the one or more recordings. The frequency spectrum may be generated using a Fourier transformation method and python libraries. The python libraries may be one of scipy.io.wavfile, scipy.ffpack import fft, Numpy and matplotlib, and the like. In one aspect, the library scipy.io.wavfile may be imported so that a python code can easily access the recording in ‘.WAV’ format. Further, the library scipy.ffpack import fft may be imported to apply fast Fourier transformation to the recording. The library Numpy and matplotlib may be used for plotting.

Further, the analysing module 216 may analyse the voice data and the frequency spectrum. The voice data and the frequency spectrum may be analysed using one of a spectral density, a coherence, a bandwidth, a signal to noise ratio, an impedance and the like. Based on the analysis, the analysis module 216 may generate analysed data. In one aspect, an audio signal associated with the voice data may be analysed. In one aspect, the frequency spectrum and the voice data may be analysed together.

In one embodiment, when an energy of the audio signal is concentrated around a finite time interval, especially if its total energy is finite, the system may compute the spectral density, also referred as an energy spectral density. It is to be noted that more commonly used is a power spectral density, also referred as simply power spectrum, which applies to signals existing over all time, or over a time period large enough, especially in relation to the duration of a measurement, that it could as well have been over an infinite time interval. The power spectral density (PSD) then refers to the spectral energy distribution that may be found per unit time, since the total energy of such a signal over all time would generally be infinite. Further, summation or integration of the spectral components yields the total power, for a physical process, or a variance, in a statistical process.

Further, the coherence may be a statistic that can be used to examine the relation between two signals or data sets.

The bandwidth may be a difference between an upper frequency and a lower frequency in a continuous band of frequencies. The bandwidth may be measured in hertz, and may be calculated for a given frequency spectrum from a predictive analytics algorithm.

The Signal-to-noise ratio, abbreviated SNR or S/N, may be a measure used in science and engineering that compares a level of a desired signal to a level of background noise. The SNR may be defined as the ratio of a signal power to a noise power, often expressed in decibels. A ratio higher than 1:1, that is greater than 0 dB, indicates more signal than noise.

The impedance, also referred as an acoustic impedance, may be a measure of the ease with which sound waves travels through a particular medium.

Further, the recommendation generation module 218 may compare the analysed data with historical data. The historical data may be associated with one or more patients. The historical data may be referred as reference data, or healthy data. The one or more patients may vary in one or more parameters comprising an age group, a gender, an ethnicity, a geography and the like. Based on the comparison, the recommendation generation module 218 may check if the analysed data is in line with the healthy data. Further, based on the comparison, and the analysis of the frequency spectrum, the recommendation generation module 218 may predict a patient's health. Further, the recommendation generation module 218 generate one or more recommendations for the patient and a doctor. The one or more recommendations may be generated based on the prediction of the patient's health. The one or more recommendations may correspond to triggering a discharge process for the patient, continuing regular treatment for the patient, and changing treatment for the patient. In one aspect, the one or more recommendations may be sent to a mobile number of the patient, patient's relatives, the doctor, and other people related to the patient. Further, the doctor and the patient may take action based on the one or more recommendations.

In one embodiment, the recommendation generation module 218 may generate a report. The report may comprise a patient's name. the voice data, the one or more recommendations and the like. The report may be used by the system and the doctor to analyse the patient's health, and to take further action.

In one example, construe a patient admitted in a hospital. The patient may have to go through the process of having their voices recorded during the various stages of their treatment. The medical team may ensure that the frequency spectrum generated by the patient's voice has to lie within range of the sampled “Healthy Data”. It may help to ensure that the patient is released on data in-line with that of healthy individuals of the same age group.

Exemplary embodiments discussed above may provide certain advantages. Though not required to practice aspects of the disclosure, these advantages may include those provided by the following features.

Some embodiments of the system and the method is configured to generate recommendations for a patient and a doctor.

Some embodiments of the system and the method is configured to predict a patient's health.

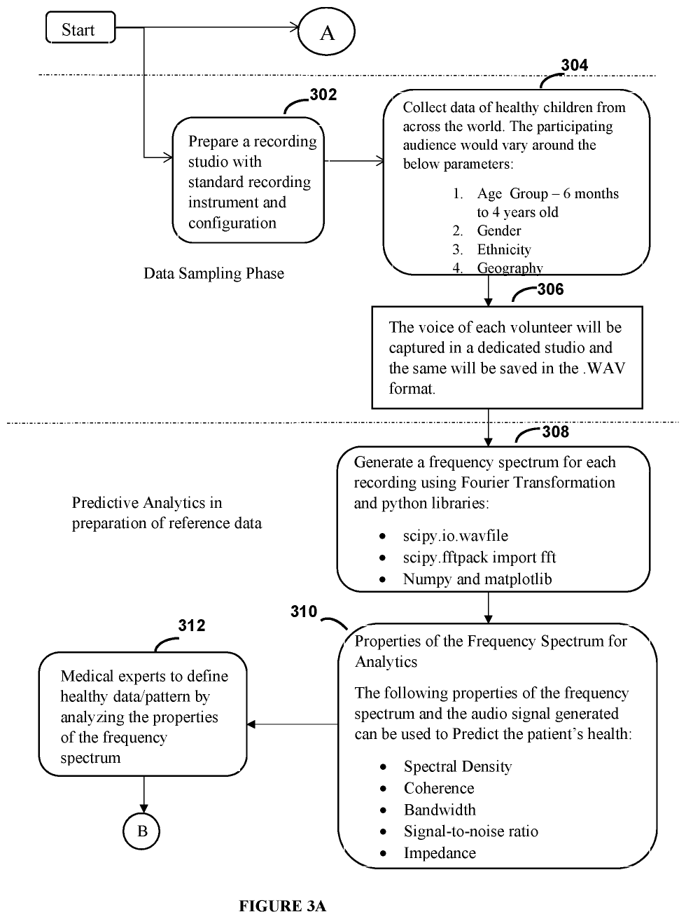

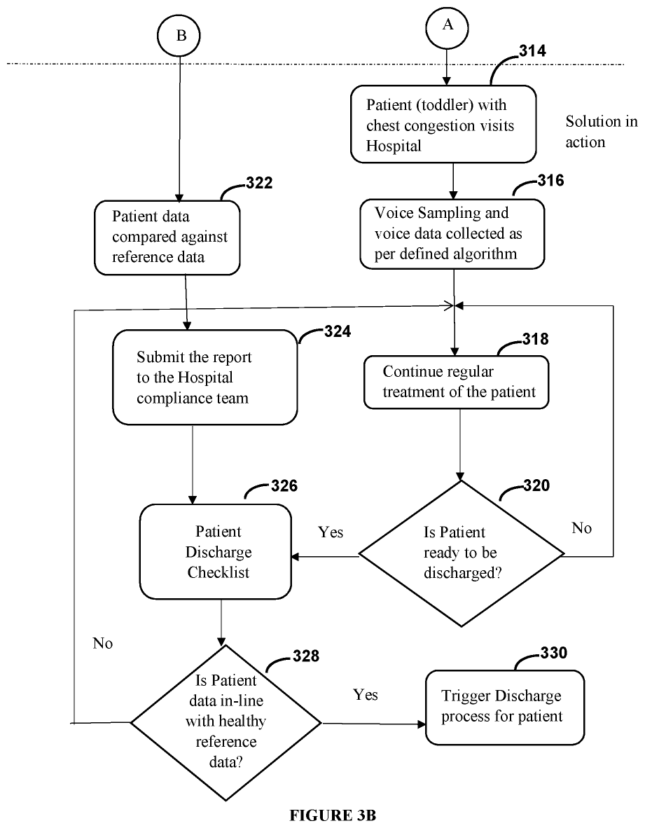

Referring now to FIG. 3A and FIG. 3B, a method for generating recommendations, is disclosed in accordance with an embodiment of the present subject matter. The method may be described in the general context of computer executable instructions. Generally, computer executable instructions can include routines, programs, objects, components, data structures, procedures, modules, functions, and the like, that perform particular functions or implement particular abstract data types. The method may also be practiced in a distributed computing environment where functions are performed by remote processing devices that are linked through a communications network. In a distributed computing environment, computer executable instructions may be located in both local and remote computer storage media, including memory storage devices.

The order in which the method is described is not intended to be construed as a limitation, and any number of the described method blocks can be combined in any order to implement the method or alternate methods. Additionally, individual blocks may be deleted from the method without departing from the spirit and scope of the subject matter described herein. Furthermore, the method can be implemented in any suitable hardware, software, firmware, or combination thereof. However, for ease of explanation, in the embodiments described below, the method may be considered to be implemented in the above described system 102.

At block 302, a recording studio may be prepared. The recording studio may use a standard instrument with standard configurations to record an audio.

At block 304, data from one or more patients may be collected. The one or more patients may be children. The data may be referred as historical data. The one or more patients may vary in one or more parameters comprising an age group, a gender, am ethnicity, a geography and the like. The historical data may be recorded in the recording studio. The historical data may correspond to historical recordings associated with the one or more patients. The historical data may be saved in ‘.WAV’ format at block 306.

At block 308, a frequency spectrum associated with each recording from the recordings in the historical data may be generated. The frequency spectrum may be generated using a Fourier transformation method and python libraries.

At block 310, the frequency spectrum and the historical data may be analysed based on one or more of a spectral density, a coherence, a bandwidth, a signal to noise ratio, an impedance. Based on the analysis, the reference data may be defined at block 312. The reference data may be healthy data that is used for further predictions associated with patients.

At block 314, voice data associated with a patient may be captured in real-time. The voice data may comprise one or more recordings associated with the patient. Upon capturing the voice data, at block 316, the voice data may be analysed, and a frequency spectrum for each recording in the voice data may be generated. In other words, a voice sampling of the voice data may be performed. The frequency spectrum associated with each recording in the voice data may be generated using a Fourier transformation method and python libraries. Further, the frequency spectrum and the voice data may be analysed based on one or more of a spectral density, a coherence, a bandwidth, a signal to noise ratio, an impedance.

At block 322, analysed data, generated based on analysis of the voice data and the frequency spectrum, may be compared with the reference data. Based on the comparison, and the frequency spectrum, a report may be generated at block 324. The repot may comprise one or more recommendations for the patient and the doctor. The report may be submitted to a hospital compliance team. At block 326, the report may be analysed to check the patient discharge checklist. At block 328, if the healthy data and the analysed data are in line with each other, then a discharge process may get triggered for the patient at block 330. If the analysed data of the patient is not in line with the healthy data, then the patient may be continued with a regular treatment at block 318.

At block 320, it may check if the patient is ready for a discharge. If the patient id ready for the discharge, then the discharge process may be triggered. If the Patient is not ready for the discharge, the regular treatment for the patient may be continued.

Although implementations for systems and methods for generating an Omni-channel support platform have been described, it is to be understood that the appended claims are not necessarily limited to the specific features or methods described. Rather, the specific features and methods are disclosed as examples of implementations for generating the Omni-channel support platform.

1. A method for generating recommendations for a patient and a doctor, the method comprising:
capturing, by a processor, data associated with a health of a patient, wherein the data comprises voice data comprising one or more recordings of the patient;
generating, by the processor, a frequency spectrum for each recording from the one or more recordings, wherein the frequency spectrum is generated using a Fourier transformation method and python libraries;
analyzing, by the processor, the voice data and the frequency spectrum based on one of a spectral density, a coherence, a bandwidth, a signal to noise ratio, and an impedance, wherein analysed data is generated based on the analysis; and
generating, by the processor, one or more recommendations for the patient and the doctor based on comparing the analysed data with historical data associated with one or more patients, and the frequency spectrum.

2. The method as claimed in claim 1, further comprises predicting a patient's health based on the frequency spectrum and the analysed data.

3. The method as claimed in claim 1, further comprises generating a report comprising a patient's name, the data, and the recommendations associated with the patient.

4. The method as claimed in claim 1, wherein the one or more recommendations corresponds to triggering a discharge process for the patient, continuing regular treatment for the patient, and changing treatment for the patient.

5. The method as claimed in claim 1, wherein the one or more recordings are recorded using a recording studio.

6. The method as claimed in claim 1, wherein the one or more patients varies in one or more parameters corresponding to an age group, a gender, an ethnicity, and a geography.

7. A system for generating recommendations for a patient and a doctor, the system comprising:
a memory;
a processor coupled to the memory, wherein the processor is configured to execute instructions stored in the memory to:
capture data associated with a health of a patient, wherein the data comprises voice data comprising one or more recordings of the patient;
generate a frequency spectrum for each recording from the one or more recordings, wherein the frequency spectrum is generated using a Fourier transformation method and python libraries;
analyze the voice data and the frequency spectrum based on one of a spectral density, a coherence, a bandwidth, a signal to noise ratio, and an impedance, wherein analysed data is generated based on the analysis; and
generate one or more recommendations for the patient and the doctor based on comparing the analysed data with historical data associated with one or more patients, and the frequency spectrum.

8. The system as claimed in claim 7, further configured to predict a patient's health based on the frequency spectrum and the analysed data.

9. The system as claimed in claim 7, further configured to generate a report comprising a patient's name, the data, and the recommendations associated with the patient.

10. The system as claimed in claim 7, wherein the one or more recommendations corresponds to triggering a discharge process for the patient, continuing regular treatment for the patient, and changing treatment for the patient.

11. The system as claimed in claim 7, wherein the one or more recordings are recorded using a recording studio.

12. The system as claimed in claim 7, wherein the one or more patients varies in one or more parameters corresponding to an age group, a gender, an ethnicity, and a geography.
